(19) United States
(12) Patent Application Publication
(10) Pub. No.: US 2024/0099799 A1
(43) Pub. Date: Mar. 28, 2024

(54) Sterile Sheath with Access Port

(71) Applicant: Bard Access Systems, Inc., Salt Lake City, UT (US)

(72) Inventors: Kelton John Michael Stokes, Salt Lake City, UT (US); Stefan Josef Fellner, Salt Lake City, UT (US)

(21) Appl. No.: 18/373,727

(22) Filed: Sep. 27, 2023

Related U.S. Application Data
(60) Provisional application No. 63/410,805, filed on Sep. 28, 2022.

(51) Int. Cl. A61B 50/30

(52) U.S. Cl. CPC A61B 50/30

(57) ABSTRACT

A sterile sheath configured to enshroud a non-sterile medical device. Certain non-sterile medical equipment, e.g., consoles, computing devices, etc. are not easily sterilizable without damaging the device itself. As such, these non-sterile medical devices remain outside of the sterile field. Engaging sterile medical devices that are within the sterile field with these non-sterile medical devices can compromise the sterility of the sterile field. Disclosed herein is a sheath configured to enshroud a portion of the non-sterile medical device. Further the sheath can include an access port to allow one or more sterile medical devices to engage the non-sterile medical device disposed within the sheath.

### PRIORITY

This application claims the benefit of priority to U.S. Provisional Application No. 63/410,805, filed Sep. 28, 2022, which is incorporated by reference in its entirety into this application.

### SUMMARY

Briefly summarized, embodiments disclosed herein are directed to apparatus and methods for a sterile sheath configured to enshroud a non-sterile medical device. Within a sterile environment, various sterile medical devices can be brought into the sterile field. These sterile medical devices tend to be either disposable and/or cleanable in order to be re-sterilized and reused. However, certain non-sterile medical equipment, e.g., consoles, computing devices, etc. are not easily sterilizable without damaging the device itself, and/or are too expensive to be a single-use item. As such, these non-sterile medical devices remain outside of the sterile field.

Situations can arise where non-sterile medical devices need to engage or communicate with sterile medical devices within the sterile field, but without compromising the sterile field. For example, in order to communicate wirelessly, the non-sterile medical device needs to be proximate the patient or proximate a sterile medical device. Alternatively, for wired communication, the non-sterile medical device needs to engage (e.g., be connected to) the sterile medical device directly.

Disclosed herein is a sheath for enshrouding a non-sterile medical device including, a body defining an elongate cavity and communicating with an opening at a first end, and a port disposed at a second end of the body, opposite the first end, and defining an aperture and including a cap selectively removable from the aperture to allow a portion of a sterile medical device to pass therethrough.

In some embodiments, the body is formed of a first material that is a flexible, gas-impervious material.

In some embodiments, the body includes a sterile surface on a first side and a non-sterile surface on a second side, opposite the first side.

In some embodiments, the body is configured to pass through the opening at the first end to transition the body between a first configuration where the non-sterile surface is disposed outwards, and a second configuration where the sterile surface is disposed outwards.

In some embodiments, the sheath further includes a first adhesive layer disposed on at least a portion of the first side and configured to secure the sheath to a portion of a sterile field.

In some embodiments, the first adhesive layer extends from the port to the opening at the first end.

In some embodiments, the sheath further includes a release layer disposed on the first adhesive layer.

In some embodiments, the sheath further includes a second adhesive layer disposed on at least a portion of the second side and configured to releasably secure a portion of the sheath to the non-sterile medical device.

In some embodiments, the port is formed of a second material that is relatively more rigid than the first material.

In some embodiments, the second material includes a plastic, polymer, rubber, or silicone rubber.

In some embodiments, one or both of the cap and the aperture of the port includes an adhesive layer configured to releasably secure the cap over the aperture and provide a seal therebetween.

In some embodiments, the cap engages the aperture in one of a threadable engagement, press-fit engagement, interference fit engagement, snap-fit engagement to releasably secure the cap over the aperture and provide a seal therebetween.

Also disclosed is a method of securing a non-sterile medical device within a sterile field including, providing a sheath having an elongate body defining a cavity and extending to an opening disposed at a first end, the sheath including a non-sterile surface disposed on a first side of the body, and a sterile surface disposed on a second side of the body, inverting the elongate body to a first configuration by urging the body through the opening such that the non-sterile surface is disposed outwards, engaging a portion of the non-sterile surface with the non-sterile medical device, inverting the elongate body from the first configuration to a second configuration where the sterile surface is disposed outwards, and adhering a portion of the second side of the body to the sterile field.

In some embodiments, the method further includes removing a cap from a port aperture disposed at a second end of the sheath, opposite the opening at the first end, to provide access to the non-sterile medical device disposed within the cavity.

In some embodiments, the method further includes extending a portion of a sterile medical device through the port aperture and securing the cap to the port aperture, the portion of the sterile medical device passing between cap and a wall of the aperture and sealed thereto to maintain a barrier between the non-sterile medical device and the sterile field.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a sheath in an exemplary environment of use, in accordance with embodiments disclosed herein;

FIG. 2A shows a sheath in a first configuration, in accordance with embodiments disclosed herein;

FIG. 2B shows a sheath in a second configuration, in accordance with embodiments disclosed herein; and

FIGS. 3A-3C shows an exemplary method of use for a sheath, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise. Labels such as “left,” “right,” “upper”, “lower,” “top,” “bottom,” “front,” “back,” and the like are used for convenience and are not intended to imply, for example, any particular fixed location, orientation, or direction. Instead, such labels are used to reflect, for example, relative location, orientation, or directions.

In the following description, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

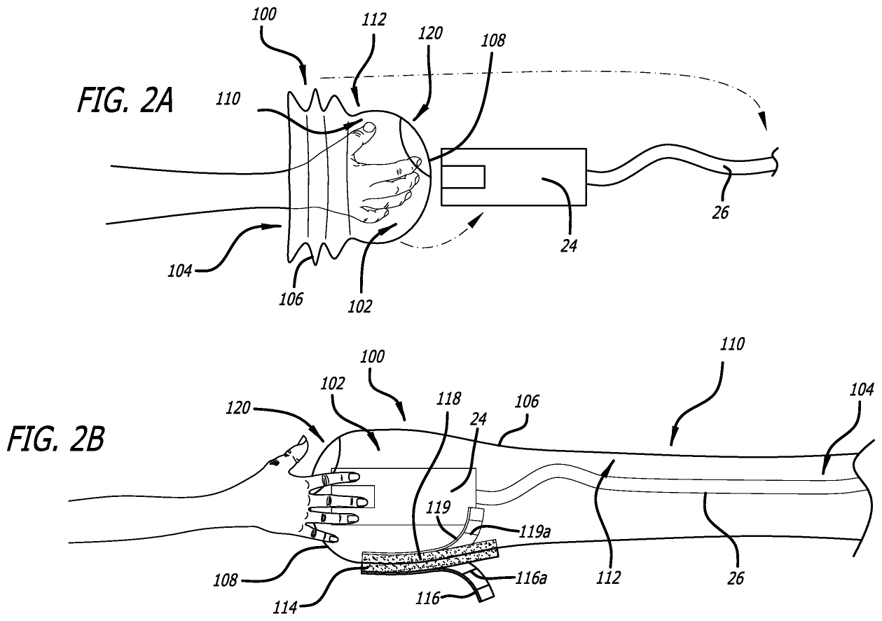

To assist in the description of embodiments described herein, a coordinate axis indication is shown in FIG. 2B, where a longitudinal axis of the sheath extends parallel to an axial length of the sheath cavity, a lateral axis extends normal to the longitudinal axis, and a transverse axis extends normal to both the longitudinal and the lateral axes.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

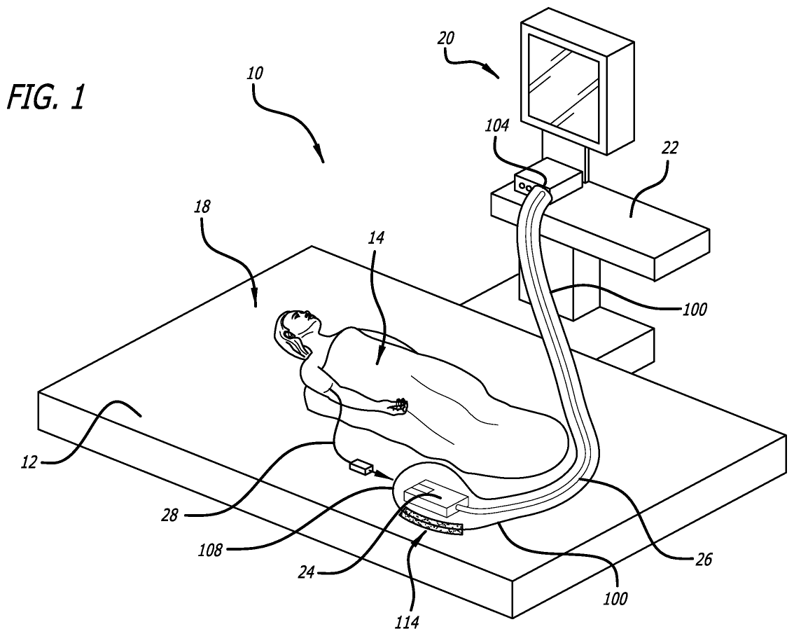

FIG. 1 shows and exemplary environment of use 10 for a sterile sheath (“sheath”) 100, as disclosed herein. Exemplary environments of use 10 can include operating tables, surgical theaters, sterile drapes, or any area where a sterile field is established. As shown, the exemplary environment of use 10, while not intended to be limiting, can include a sterile field 18 which may include an operating table 12 having a patient 14 disposed thereon. The exemplary environment of use 10 can include one or more pieces of sterile medical equipment 28 that can enter (i.e., be disposed within) the sterile field 18. Exemplary sterile medical equipment 28 can include, but not limited to, catheters, needles, stylets, scalpels, gauze, drapes, and the like. The exemplary environment of use 10 can further include one or more pieces of non-sterile medical equipment, e.g. a console 22, disposed outside of the sterile field 18, i.e., within a non-sterile field 20.

In an embodiment, one or more pieces of non-sterile medical equipment, e.g., a connector 24 and/or wire harness 26, may need to extend into the sterile field 18 to be disposed proximate the patient 14 in order to function correctly. For example, the connector 24 may include a wireless antenna that needs to be within range of a sterile wireless medical device 28 within the sterile field 18. Alternatively, a sterile medical device 28 may need to engage the non-sterile connector 24 to establish wired communications therebetween. Advantageously, the sheath 100 can enshroud portions of the non-sterile medical devices, e.g., the connector 24, to maintain a barrier between the non-sterile connector 24 and the sterile field 18. As such, the non-sterile connector 24 can be placed proximate the patient 14 without compromising the sterile field 18. It will be appreciated that the non-sterile connector 24 and/or wire harness 26 are exemplary and not intended to be limiting. As such, the sheath 100 can be configured to enshroud various non-sterile probes, ultrasound probes, sensors, connection modules, communication modules, and the like in order to allow these devices to enter the sterile field 18 without compromising the sterility of the sterile field 18.

FIGS. 2A-2B show further details of the sheath 100. In an embodiment, the sheath 100 can include an elongate body (or wall) 106 defining a cavity 102 extending axially along a longitudinal axis. The body 106 can be formed of a first material, for example a thin, flexible, gas-impervious material such as a woven or non-woven, transparent, semi-transparent or opaque, plastic, polymer, polyurethane, combinations thereof, or the like. The cavity 102 can communicate with an opening 104 disposed at a first end. The opening 104 can be disposed outside of the sterile field 18. The body 106 of the sheath 100 can extend from the opening 104 at the first end to a base 108 disposed at a second end, opposite the first end. Further, the body 106 of the sheath 100 can include a sterile surface 110 disposed on a first side of the body 106, and a non-sterile surface 112 disposed on a second side of the body 106, opposite from the first side.

As shown in FIGS. 2A-2B, the sheath 100 can be invertible between a first configuration (FIG. 2A) and a second configuration (FIG. 2B). As shown in FIG. 2A, the body 106 can be urged through the opening 104 such that the non-sterile surface 112 is facing outwards and the sterile surface 110 is facing inwards, into the cavity 102 and as such creates a sterile cavity 102. As shown in FIG. 2B, the sheath 100 can be transitioned from the first configuration to the second configuration by urging the body 106 through the opening 104 such that the sterile surface 110 is facing outwards and the non-sterile surface 112 is facing inwards, and as such creates a non-sterile cavity 102.

In an exemplary method of use, the sheath 100 can be provided, as described herein in a first configuration (FIG. 2A). A clinician can place a sterile hand through the opening 104 and into the sterile cavity 102 where the sterile surface 110 of the body 106 is facing inwards into the cavity 102. The clinician can then grasp the non-sterile connector 24 by contacting the non-sterile surface 112 of the body 106, disposed outwards, with the non-sterile connector 24.

The clinician can then invert the sheath 100 from the first configuration to the second configuration to surround the non-sterile connector 24 with the sheath 100. In the second configuration, the sheath 100 defines a non-sterile cavity 102 having the non-sterile surface 112 facing inwards. The opening 104 can then be placed outside of the sterile field 18, while the non-sterile connector 24 that is contained within the cavity 102 can be placed within the sterile field 18 without compromising the sterile field 18 (FIG. 1).

In an embodiment, the sheath 100 can include a first adhesive layer 114 disposed on a portion of the sterile surface 110, e.g., adjacent the connector 24. In an embodiment, the adhesive layer 114 can extend along an entire length of the sheath 100 from base end 108 to the opening 104. In an embodiment, the adhesive layer 114 can include a release layer 116 disposed thereon. Peeling back a portion of the release layer 116 can expose a portion of the adhesive layer 114 disposed therebelow and can allow the user to adhere the sheath 110, including the connector 24 disposed therein, to the sterile field 18, e.g., a portion thereof or a sterile object within the sterile field 18. Advantageously, the first adhesive layer 114 can mitigate the sheath 100 and connector 24 assembly from sliding out of the sterile field 18. In an embodiment, the first adhesive layer 114 can be a relatively low-tac adhesive configured to allow the clinician to attached and detach the sheath 100 from the sterile field 18, as needed. In an embodiment, the release layer 116 can include one or more tear lines 116A, such as a perforation, laser cut line, score line etc. configured to allow a first portion of the release layer 116 to be removed while allowing a second portion of the release layer 116 to remain in place. As such, the user can selectively expose different portion(s) of the first adhesive layer 114 and adhere these portion(s) to the sterile field 18 as needed, while the remaining portions of the first adhesive layer 114 remain covered.

In an embodiment, the sheath 100 can include a second adhesive layer 118 disposed on a portion of the non-sterile surface 112 and configured to releasably engage a non-sterile medical device, such as portion of one or both of the connector 24 and the wire harness 26, for example, to secure the sheath 100 thereto. Optionally, the second adhesive layer 118 can also include a release layer 119 similar to the release layer 116, disposed on the second adhesive layer 118. Advantageously, the release layer 116 disposed on the second adhesive layer 118 can be selectively removed therefrom depending on if the clinician would like to further secure the connector 24 within the cavity 102, or if the clinician would prefer the connector 24 to move freely within the cavity 102. Similar to the release layer 116, the releases layer 119 may include one or more tear lines 119A

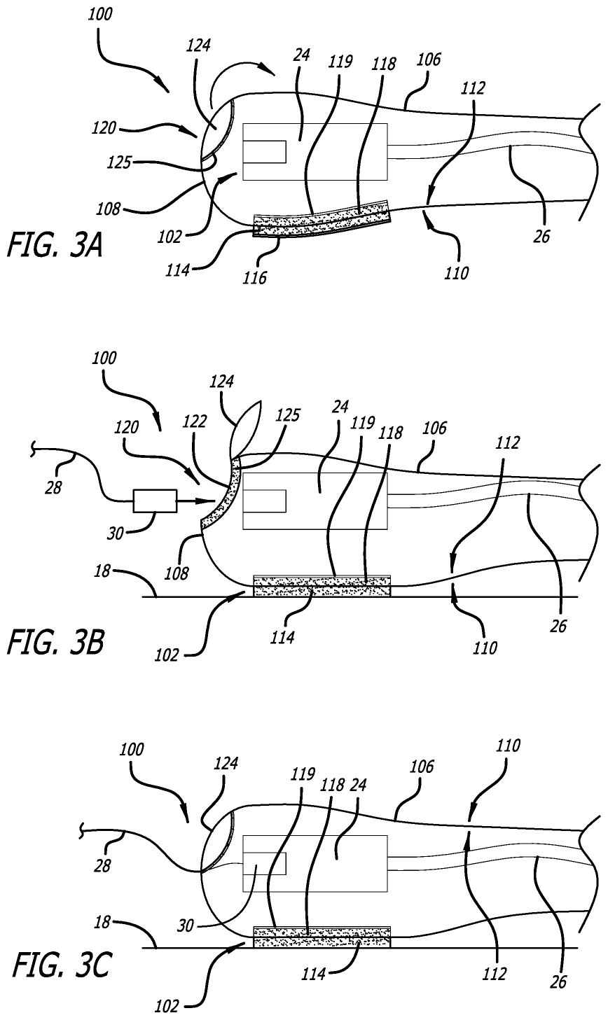

As shown in FIGS. 3A-3C, in an embodiment, the sheath 100 can further include a port 120 disposed at a base end 108 of the sheath 100, opposite to the opening 104. The port 120 can generally include a port aperture 122 (see FIG. 3B) and a cap 124. In an embodiment, one or both of a wall defining the port aperture 122 and the cap 124 can be formed of the same material as the body 106, i.e., the first material. The cap 124 can cover the port aperture 122 (as shown in FIG. 3A) to maintain the integrity of the sterile barrier formed by the sheath 100. The cap 124 can transition between an open configuration and a closed configuration to allow a clinician to selectively access the connector 24 through the port aperture 122 of the port 120.

In an embodiment, one or both of the cap 124 and the wall defining the port aperture 122 can include an adhesive layer 125 to releasably secure the cap 124 over the port aperture 122. The adhesive of the adhesive layer 125 can be a relatively low-tac adhesive configured to allow the cap 124 to attach to, and detach from, the port aperture 122.

In an embodiment, a portion of a sterile medical device 28 can extend through the port aperture 122 to engage the non-sterile connector 24 disposed within the non-sterile cavity 102. In an embodiment, a portion of the cap 124 can engage the port aperture 122 and can flexibly deform around the portion of the sterile medical device 28 extending through the port aperture 122 to provide a seal.

In an embodiment, the port 120 can be formed of a second material, different from that of the first material of the sheath body 106. In an embodiment, the port 120 can be formed of a flexible, silicone rubber material. Further, the cap 124 can be molded into a bi-stable shape allowing the cap to remain in either the open configuration or the closed configuration and can be transitioned therebetween. Further, the cap 124 can flexibly deform around the portion of the sterile medical device 28 extending through the port aperture 122 to provide a seal, as described herein.

In an embodiment, the port 120 can be formed of a relatively rigid material and can define the port aperture 122 and the cap 124. The cap 124 can be selectively attached over the port aperture 122 using a threadable engagement, press-fit, interference fit, snap-fit engagement, combinations thereof, or the like. In an embodiment, the cap 124 can be hingedly coupled to the port aperture 122 with either a living hinge or a mechanical hinge. In an embodiment, the cap 124 can further include a tab, handle, finger loop, or similar gripping feature, extending therefrom and configured to facilitate grasping of the cap 124.

In an exemplary method of use, as shown in FIGS. 3A-3C, a connector 24 or similar non-sterile medical device can be placed within the sheath 100, as described herein (FIGS. 2A-2B). Exemplary non-sterile medical devices can include, but not limited to, a connector 24 and wire harness 26 for an ultrasound imaging device, a tracking device for an intra-corporeal or intra-vascular medical device, a patient attribute recording device configured to record pulse rate, breathing rate, blood pressure, ECG, body temperature, etc., or similar console 20 disposed outside of the sterile field 18. Once disposed within the sheath 100, the connector 24 can be placed proximate the patient 14 without compromising the sterile field 18.

In an embodiment, a clinician may want to selectively couple a sterile medical device 28 to the connector 24. Exemplary sterile medical devices 28 can include, but not limited to, an ultrasound probe, a sensor enable intravascular medical device such as a catheter, guidewire, or stylet, electrodes, combinations thereof, or the like. As such, as shown in FIG. 3A, the clinician can selectively remove the cap 124 exposing the port aperture 122 therebelow. As shown in FIG. 3B, the clinician can then advance a sterile contact 30 of the sterile medical device 28 through the port aperture 122 and engage the connector 24 disposed within the sheath 100.

As shown in FIG. 3C, with a portion of the sterile medical device 28 extending through the port aperture 122, the clinician can then close the cap 124 over the port aperture 122 and provide a seal around the portion of the sterile medical device 28 extending through the port aperture 122. For example, where the cap 124 is formed of a relatively flexible material, the cap 124 can flexibly deform around the portion of the sterile medical device 28. Optionally, an adhesive layer can secure the cap 124 about the portion of the sterile medical device 28. In an embodiment, where the cap 124 is formed of a more rigid material, the cap 124 can include a notch, gasket, bearing, valve, or similar structure configured to allow the portion of the sterile medical device 28 to extend therethrough while maintaining a barrier between the sterile field 18 and the non-sterile connector 24. Optionally, the sheath 100 can then be adhered in place using the first adhesive layer 114 disposed on the sterile surface 112. Advantageously, the sheath 100 can allow a clinician to access non-sterile medical devices within the sterile field 18 without compromising the sterility of the sterile field 18.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

what is claimed is:

1. A sheath for enshrouding a non-sterile medical device, comprising:
a body defining an elongate cavity and communicating with an opening at a first end; and
a port disposed at a second end of the body opposite the first end, the port including an aperture and a cap selectively removable from the aperture to allow a portion of a sterile medical device to pass therethrough.

2. The sheath according to claim 1, wherein the body is formed of a first material that is a flexible, gas-impervious material.

3. The sheath according to claim 1, wherein the body includes a sterile surface on a first side and a non-sterile surface on a second side, opposite the first side.

4. The sheath according to claim 3, wherein the body is configured to pass through the opening at the first end to transition the body between a first configuration where the non-sterile surface is disposed outwards, and a second configuration where the sterile surface is disposed outwards.

5. The sheath according to claim 3, further including a first adhesive layer disposed on at least a portion of the first side and configured to secure the sheath to a portion of a sterile field.

6. The sheath according to claim 5, wherein the first adhesive layer extends from the port to the opening at the first end.

7. The sheath according to claim 5, further including a release layer disposed on the first adhesive layer.

8. The sheath according to claim 3, further including a second adhesive layer disposed on at least a portion of the second side and configured to releasably secure a portion of the sheath to the non-sterile medical device.

9. The sheath according to claim 2, wherein the port is formed of a second material that is relatively more rigid than the first material.

10. The sheath according to claim 9, wherein the second material includes a plastic, polymer, rubber, or silicone rubber.

11. The sheath according to claim 1, wherein one or both of the cap and the aperture of the port includes an adhesive layer configured to releasably secure the cap over the aperture and provide a seal therebetween.

12. The sheath according to claim 1, wherein the cap engages the aperture in one of a threadable engagement, press-fit engagement, interference fit engagement, snap-fit engagement to releasably secure the cap over the aperture and provide a seal therebetween.

13. A method of securing a non-sterile medical device within a sterile field, comprising:
providing a sheath having an elongate body defining a cavity and extending to an opening disposed at a first end, the sheath including a non-sterile surface disposed on a first side of the body, and a sterile surface disposed on a second side of the body;
inverting the elongate body to a first configuration by urging the body through the opening such that the non-sterile surface is disposed outwards;
engaging a portion of the non-sterile surface with the non-sterile medical device;
inverting the elongate body from the first configuration to a second configuration where the sterile surface is disposed outwards; and
adhering a portion of the second side of the body to the sterile field.

14. The method according to claim 13, further including removing a cap from a port aperture disposed at a second end of the sheath, opposite the opening at the first end, to provide access to the non-sterile medical device disposed within the cavity.

15. The method according to claim 14, further including extending a portion of a sterile medical device through the port aperture and securing the cap to the port aperture, the portion of the sterile medical device passing between cap and a wall of the aperture and sealed thereto to maintain a barrier between the non-sterile medical device and the sterile field.
